(19) United States
(12) Patent Application Publication
(10) Pub. No.: US 2010/0249598 A1
(43) Pub. Date: Sep. 30, 2010

(54) ULTRASOUND PROBE WITH REPLACEABLE HEAD PORTION

(75) Inventors: Lowell Scott Smith, Niskayuna, NY (US); Charles Edward Baumgartner, Niskayuna, NY (US); Charles Gerard Woychik, Niskayuna, NY (US); Warren Lee, Niskayuna, NY (US); Reinhold Bruestle, Zipf (AT); Ferdinand Puttinger, Zipf (AT)

Correspondence Address: GENERAL ELECTRIC COMPANY;GLOBAL RESEARCH, ONE RESEARCH CIRCLE, BLDG. K1-3A59, NISKAYUNA, NY, 12309, US

(73) Assignee: GENERAL ELECTRIC COMPANY, Schenectady, NY (US)

(21) Appl. No.: 12/410,525

(22) Filed: Mar. 25, 2009

(51) Int. Cl. A61B 8/13; H04R 31/00

(52) U.S. Cl. 600/459; 29/594

(57) ABSTRACT

An ultrasound probe includes a transducer comprising an array of transducer elements removably disposed in a head portion. At least one or more stages of electronic circuit units is removably coupled to the transducer and configured to excite the transducer. A handle portion is detachably coupled to the head portion. The head portion and the handle portion are disposed enclosing the at least one or more stages of electronic circuit units. The ultrasound probe is used for one dimensional applications, two dimensional applications, and volumetric applications.

### BACKGROUND

The invention relates generally to an ultrasound probe, and more particularly to an ultrasound probe having a replaceable head portion.

Various noninvasive diagnostic imaging modalities are capable of producing cross-sectional images of organs or vessels inside the body. An imaging modality that is well suited for such noninvasive imaging is ultrasound. Ultrasound diagnostic imaging systems are in widespread use by cardiologists, obstetricians, radiologists and others for examinations of the heart, a developing fetus, internal abdominal organs and other anatomical structures. These systems operate by transmitting waves of ultrasound energy into the body, receiving ultrasound echoes reflected from tissue interfaces upon which the waves impinge, and translating the received echoes into structural representations of portions of the body through which the ultrasound waves are directed.

In conventional ultrasound imaging, objects of interest, such as internal tissues and blood, are scanned using planar ultrasound beams or slices. A linear array transducer is conventionally used to scan a thin slice by narrowly focusing the transmitted and received ultrasound in an elevated direction and steering the transmitted and received ultrasound throughout a range of angles in an azimuth direction. A transducer having a linear array of transducer elements, which is also known as a one-dimensional array, can operate in this manner to provide a two-dimensional image representing a cross-section through a plane that is perpendicular to a face of the transducer.

Linear arrays can also be used to generate three-dimensional images, which are also known as “volumetric” images, by translating the one-dimensional array linearly in the elevated direction or by sweeping the array through a range of angles extending in the elevated direction. Volumetric ultrasound images can also be conventionally obtained by using a two-dimensional array transducer to steer the transmitted and received ultrasound about two axes.

A conventional ultrasound probe assembly includes a system connector, cabling, and a transducer. These conventional ultrasound probes are designed and manufactured for use in specific applications. In other words for example, different ultrasound probes are required for scanning different parts of the body. The requirement of different probes for different applications increases the amount of cabling and electronic circuitry that needs to be duplicated in each probe, thereby leading to higher costs for the manufacturer and end user. In addition, portability for compact systems such as laptop-based ultrasound systems is reduced due to the need for carrying multiple bulky probe assemblies. Also, the downtime is increased. When a probe is damaged, the entire probe would need to be replaced.

There is a need for an ultrasound probe that is partly replaceable and suitable for wide variety of applications.

### BRIEF DESCRIPTION

In accordance with an exemplary embodiment of the present invention, an ultrasound probe includes a transducer comprising an array of transducer elements removably disposed in a head portion. At least one or more stages of electronic circuit units is coupled to the transducer and configured to excite the transducer. A handle portion is detachably coupled to the head portion. The head portion and the handle portion are disposed enclosing the at least one or more stages of electronic circuit units. The ultrasound probe is used for one dimensional applications, two dimensional applications, and volumetric applications.

In accordance with another exemplary embodiment of the present invention, a transducer stack assembly for an ultrasound probe includes a piezoelectric transducer layer disposed between the at least one acoustic matching layer and a dematching layer. The dematching layer is disposed on an interposer layer. The interposer layer is disposed between the dematching layer and an integrated circuit.

In accordance with another exemplary embodiment of the present invention, a transducer stack assembly for an ultrasound probe includes a piezoelectric transducer layer disposed between the at least one acoustic matching layer and a dematching layer. The dematching layer is disposed on the substrate provided with conductive bumps.

In accordance with another exemplary embodiment, a method of manufacturing a transducer stack assembly for an ultrasound probe is disclosed.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical representation of an ultrasound system having a probe assembly in accordance with an exemplary embodiment of the present invention;

FIG. 2 is a diagrammatical representation of an ultrasound probe having a replaceable head portion in accordance with an exemplary embodiment of the present invention;

FIG. 3 is a diagrammatical representation of an ultrasound probe having a replaceable head portion in accordance with an exemplary embodiment of the present invention;

FIG. 4 is a diagrammatical representation of an ultrasound probe having a mechanical joint and a dielectric barrier;

FIG. 5 is a diagrammatical representation of an ultrasound probe having a replaceable head portion in accordance with an exemplary embodiment of the present invention;

FIG. 6 is a diagrammatical representation of an ultrasound probe having a replaceable head portion plugged into the handle portion in accordance with an exemplary embodiment of the present invention;

FIG. 7 is a diagrammatical representation of a transducer array of an ultrasound probe in accordance with an exemplary embodiment of the present invention;

FIG. 8 is a diagrammatical representation of a transducer array of an ultrasound probe in accordance with an exemplary embodiment of the present invention;

FIG. 9 is a diagrammatical representation of an ultrasound probe having a replaceable head portion in accordance with an exemplary embodiment of the present invention;

FIG. 10 is a diagrammatical representation of an ultrasound probe having a replaceable head portion in accordance with an exemplary embodiment of the present invention; and

FIG. 11 is a diagrammatical representation of an ultrasound probe having a replaceable head portion in accordance with an exemplary embodiment of the present invention.

### DETAILED DESCRIPTION

In accordance with certain exemplary embodiments of the present invention, an ultrasound probe assembly includes a system connector, cabling, and a probe having a transducer including an array of transducer elements disposed in a head portion. At least one or more stages of electronic circuit units are coupled to the transducer and configured to excite the transducer. A handle portion is detachably coupled to the head portion. The head portion and the handle portion are disposed enclosing the at least one or more stages of electronic circuit units. In accordance with certain other embodiments of the present invention, a transducer stack assembly or method of manufacturing thereof for an ultrasound probe is disclosed. An ultrasound probe having a two-dimensional array of transducer elements and beam forming electronic circuits for volumetric scanning is designed in such a way that the transducer array and the electronic circuits are separable from the rest of the probe. The probe accepts other transducer arrays designed for different scanning applications. This minimizes the amount of cabling and electronic circuits that needs to be duplicated in each probe assembly, thereby leading to a higher performance per unit cost. The ultrasound probe may be used for one-dimensional applications, two-dimensional applications, and volumetric applications.

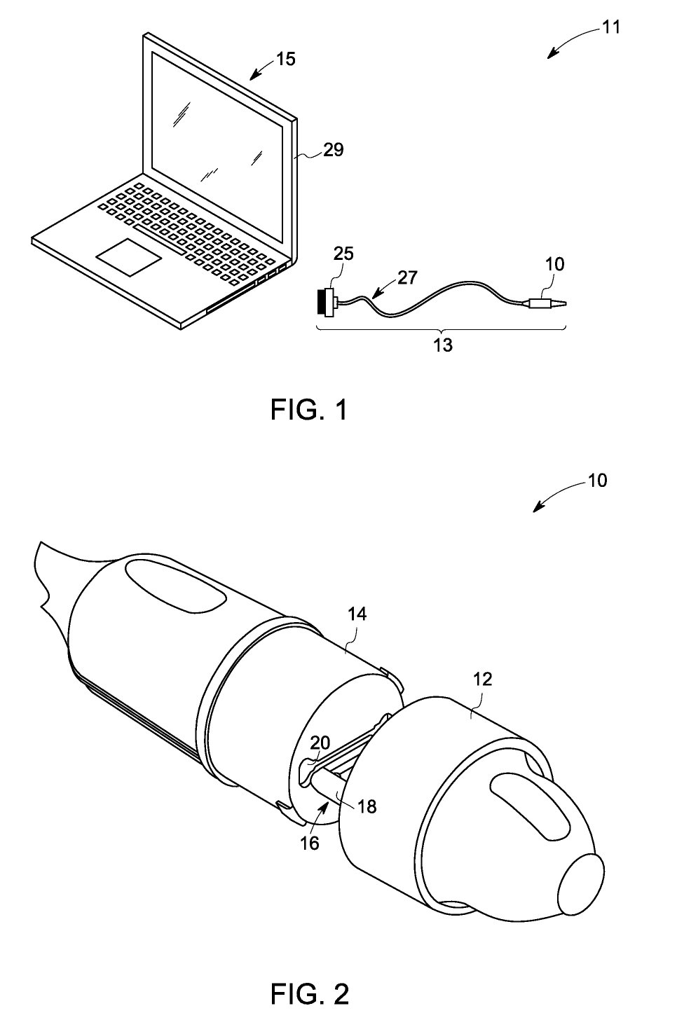

Referring to FIG. 1, an ultrasound system 11 in accordance with an exemplary embodiment of the present invention is disclosed. The ultrasound system 11 includes a probe assembly 13 and a central processing unit (CPU) 15. The probe assembly includes a transducer probe 10 coupled to a system connector 25 via a cabling 27. The system connector 25 is adapted to be coupled to the central processing unit 15. The probe 10 is configured to send and receive the sound waves. The probe 10 is explained in greater detail in the subsequent embodiments.

The CPU 15 is basically a computer that includes a microprocessor, memory, amplifiers and power supplies for the microprocessor and the probe 10. The CPU 15 sends electric currents to the transducer probe 10 to emit sound waves, and also receives the electrical pulses from the probe 10 that were created from the returning echoes. The CPU 15 performs the calculations involved in processing the data. Once the raw data is processed, the CPU 15 forms the image on a monitor 29. The CPU 15 can also store the processed data and/or image on a disk.

Referring to FIG. 2, an ultrasound probe 10 in accordance with an exemplary embodiment of the present invention is disclosed. The probe 10 includes a head portion 12 and a handle portion 14 detachably coupled to the head portion 12. In the illustrated embodiment, the head portion 12 is shown detached from the handle portion 14. Ultrasonic diagnostic imaging systems are in widespread use for performing ultrasonic imaging and measurements of the human body through the use of probes which are used to view the internal structure of a body by creating a scan plane. Ultrasound probes are generally used external to the body in non-invasive procedures but can also be used internal to the body being examined during surgical procedures. For example, the transesophageal probe (TEE Probe) is used endoscopically, for example, for ultrasonic imaging of the heart. A conventional ultrasound probe employs a one-dimensional transducer array to obtain a two-dimensional cross-sectional image of the subject's heart. Two-dimensional transducer arrays can be used to obtain a three-dimensional volumetric image. Ultrasonic transducers are also useful for various other applications. Ultrasonic testing equipment is used in a variety of applications such as for measuring flow, determining flaws, measuring thickness, and gauging corrosion.

In the illustrated embodiment, the handle portion 14 is detachably coupled to the head portion 12 via a mechanical joint 16. The mechanical joint 16 may include one or more hooks 18 provided to the head portion 12 and configured to be detachably coupled to one or more recesses 20 provided in the handle portion 14. Although, hooks 18 and recesses 20 are disclosed, other suitable mechanical joints are also disclosed. As discussed previously, different ultrasound probes are required for scanning different parts of the body. The design of the head portion 12 of the probe 10 is dependent on the subject's size and available acoustic window. Conventionally, the requirement of different probes for different applications results in connectors, cabling and electronic circuitry that needs to be duplicated for each probe assembly. The duplication of various components of the probes increases the costs associated with being able to image different applications due to the requirement of having multiple imaging probe assemblies. Furthermore, when a transducer is damaged, the entire probe would need to be replaced. Although different transducers may be required for different applications, the probe cabling and system connectors may be shared in common with the different transducer heads. In accordance with an exemplary embodiment of the present invention, the head portion 12 and desired components within the ultrasound probe 10 are replaceable since the head portion 12 is detachable from the handle portion 14. This avoids the duplication of entire probe assembly required for different scanning applications. Also, when a probe is damaged, only the required components of the probe need to be replaced instead of replacing the entire probe. Interchangeable transducer heads also results in a more compact, portable system.

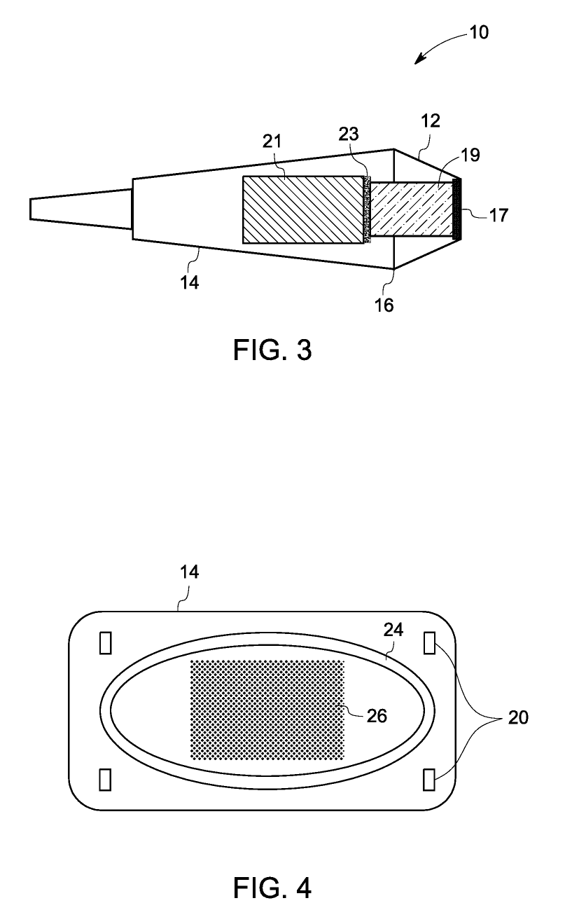

Referring to FIG. 3, an ultrasound probe 10 in accordance with an exemplary embodiment of the present invention is disclosed. As discussed previously, the probe 10 includes the head portion 12 and the handle portion 14 detachably coupled to the head portion 12. The handle portion 14 is detachably coupled to the head portion 12 via the mechanical joint 16. In the illustrated embodiment, a transducer 17 including a two-dimensional array of transducer elements (not shown) is disposed in the head portion 12. Ultrasonic transducers are used for a variety of applications, which require different characteristics. The ultrasonic transducer 17 converts electrical energy to mechanical energy and vice versa. The ultrasonic transducer 17 is constructed by incorporating one or more piezoelectric vibrators, which are electrically coupled to a pulsing-receiving system. The ultrasonic transducer 17 includes an ultrasonic transmitting/receiving element typically consisting of piezoelectric element connected to a plurality of electrodes. The ultrasound transducer 17 transmits ultrasonic waves into the tissue and receives ultrasonic echoes, which are reflected from the tissue. The transducer 17 may be placed on a body surface or inserted into a patient's body in a selected imaging region. A first stage electronic circuit unit 19 is coupled to the transducer 17 disposed in the head portion 12. A second stage electronic circuit unit 21 is removably coupled to the first stage electronic circuit unit 19 via a joint 23. The joint may include an electrical joint, mechanical joint, or combinations thereof. The modular electronic circuit units are configured to excite the transducer 17. The head portion 12 and the handle portion 14 are disposed enclosing the electronic circuit units 19, 21. It should be noted herein that depending on the design of the beam former, it might be possible to perform much of the electronic beam forming in the first stage of the electronic circuit unit 19 that no second stage electronic circuit unit will be required in the handle portion 14. It should be noted herein that the number of stages of the electronic circuit units might vary depending upon the application.

In accordance with the exemplary embodiment, different sensors can be mounted on the same handle portion depending upon the requirement/application. In other words, the head portion 12, and other components within the probe 10 are replaceable depending upon the requirement. These different sensors may operate at different central frequencies, and have different transducer pitches. The various sensors may be optimized for scanning different parts of the body, for example, pediatric vs. adult cardiology where the array architectures are similar, but since the chest, and heart sizes are different, high frequency (for example greater than 5 Megahertz) and low frequency (less than 4 Megahertz) probes are used for the respective patients. Additionally, it is possible to have a single handle portion used for different applications (for example, obstetric and peripheral vascular applications) even though the frequency and array sizes of the head portions are somewhat different. This allows a significant part of the probe to remain unchanged. Additionally, in scenarios where portions of the probes are frequently damaged during use by careless operators or accidents, only the damaged portions of the probe need to be replaced, thus reducing the repair cost incurred. Hence, using a single system connector and cable, with replaceable heads, a customer can perform a wider variety of ultrasound scanning for less total outlay.

Referring to FIG. 4, a dielectric barrier 24 in accordance with an exemplary embodiment of the present invention is disclosed. As discussed previously, the handle portion 14 is detachably coupled to the head portion via a mechanical joint. The mechanical joint may include one or more hooks provided to the head portion and configured to be detachably coupled to one or more recesses 20 provided in the handle portion 14. The dielectric barrier 24 is disposed contacting the mechanical joint. In the illustrated embodiment, the dielectric barrier 24 is an O-ring seal. An array of electric contact elements 26 of the handle portion 14 is also illustrated. During normal operation of the probe, for example, imaging operation, the handle portion 14 and the head portion are joined together mechanically. The O-ring seal would preferably be inside the mechanical joint so as to achieve a dielectric barrier between the outside and the electrical connections within the probe. This is necessary to satisfy electrical safety requirements within the probe. Although an O-ring seal is disclosed, other suitable dielectric barriers are also envisaged. In an alternate embodiment, a specialized tool would be advantageous for simultaneously depressing the appropriate parts of the mechanical joint while gently separating the head portion and handle portion 14, so as to simplify the process of replacing the head portion.

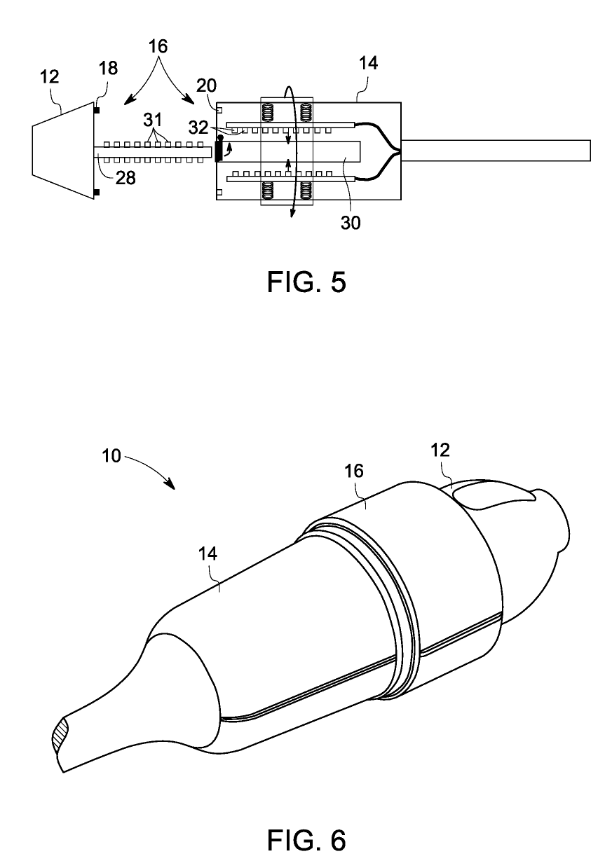

Referring to FIG. 5, an ultrasound probe 10 in accordance with an exemplary embodiment of the present invention is disclosed. In the illustrated embodiment, the head portion 12 is shown detached from the handle portion 14 detachably coupled to the head portion 12. As discussed earlier, the head portion 12, and the electronic circuit units are replaceable. In the illustrated embodiment, the head portion 12 is detached from the handle portion 14 by disengaging the mechanical joint 16. In other words, the hooks 18 of the head portion 12 is disengaged from the recesses 20 of the handle portion 14 and the head portion 12 is moved away from the handle portion 14 by rotary motion. When the head portion 12 needs to be plugged into the handle portion 14, a guide portion 28 of the head portion 12 is inserted into a guide path 30 of the handle portion 14, and the head portion 12 is moved towards the handle portion 14 until the hooks 18 are engaged to the recesses 20. A rotary motion causes a plurality of electrical contacts 31 of the head portion 12 to engage with a plurality of corresponding electrical contacts 32 of the handle portion 14. It should be noted herein the configuration of the illustrated probe is an exemplary embodiment and should not be construed in any way as limiting.

Referring to FIG. 6, an ultrasound probe 10 in accordance with an exemplary embodiment of the present invention is disclosed. In the illustrated embodiment, the head portion 12 is shown detachably coupled to the handle portion 14. When the handle portion 14 and the head portion 12 is in the plugged position, the hooks of the head portion 12 are engaged to the recesses of the handle portion 14. The dielectric barrier is disposed contacting the mechanical joint 16.

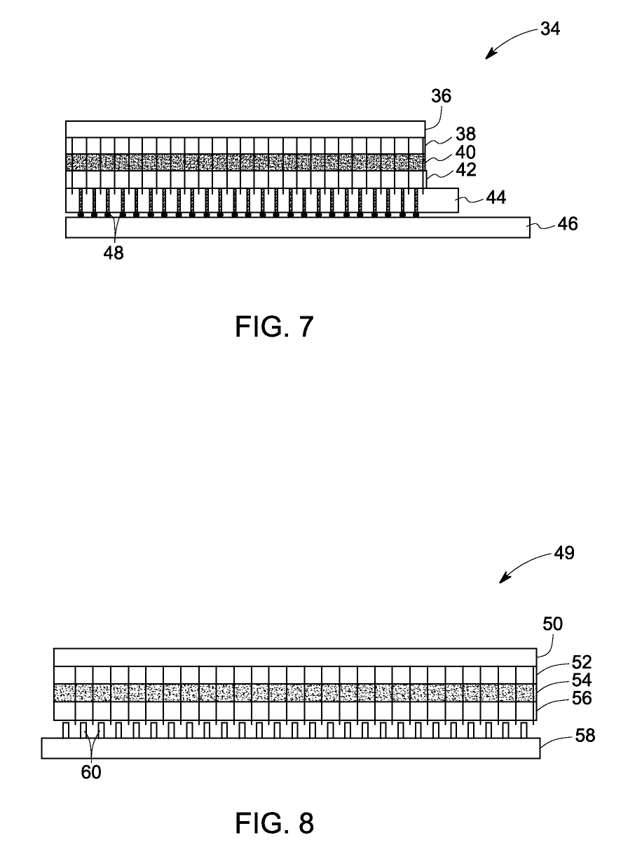

Referring to FIG. 7, a transducer array 34 in accordance with an exemplary embodiment of the present invention is disclosed. The illustrated array 34 includes two acoustic matching layers 36, 38, a piezoelectric transducer layer 40, and a dematching layer 42. The acoustic matching layer 36 is disposed on the acoustic matching layer 38. The acoustic matching layers 36, 38 are employed in ultrasound technology in order to reduce reflections outside an examination subject at boundary surfaces between two materials having different impedance, or to transmit the ultrasound energy (waves) from the transducer into the examination subject and back with as little loss as possible. In certain embodiments, this acoustic matching layers 36, 38 are diced with cuts running in the elevation dimension. The piezoelectric transducer layer 40 is disposed between the dematching layer 42 and the acoustic matching layer 38. An interposer layer 44 is disposed between the dematching layer 42 and an integrated circuit 46 having a plurality of bumps 48, which also provide a space between these two layers. The bumps 48 may include conductive bumps including gold, copper, solder, silver epoxy, or combinations thereof. The dematching layer 42 includes a conductive material with a high acoustic impedance configured to retard the coupling of acoustic energy from the piezoelectric transducer layer 40 into the integrated circuit 46 having the plurality of bumps 48. In other words, the dematching layer 42 isolates the interposer layer 44 and the integrated circuit 46 from most of the acoustic energy.

Referring to FIG. 8, a transducer array 48 in accordance with an exemplary embodiment of the present invention is disclosed. The illustrated array 48 includes two acoustic matching layers 50, 52, a piezoelectric transducer layer 54, and a dematching layer 56. The acoustic matching layer 50 is disposed on the acoustic matching layer 52. The piezoelectric transducer layer 54 is disposed between the dematching layer 56 and the acoustic matching layer 52. The dematching layer 56 is disposed on a wafer (substrate) 58 having a plurality of conductive bumps 60 including gold, copper, solder, silver epoxy, or combinations thereof, which also provide a space between these two layers. The dematching layer 56 is configured to isolate the substrate 58 from acoustic energy.

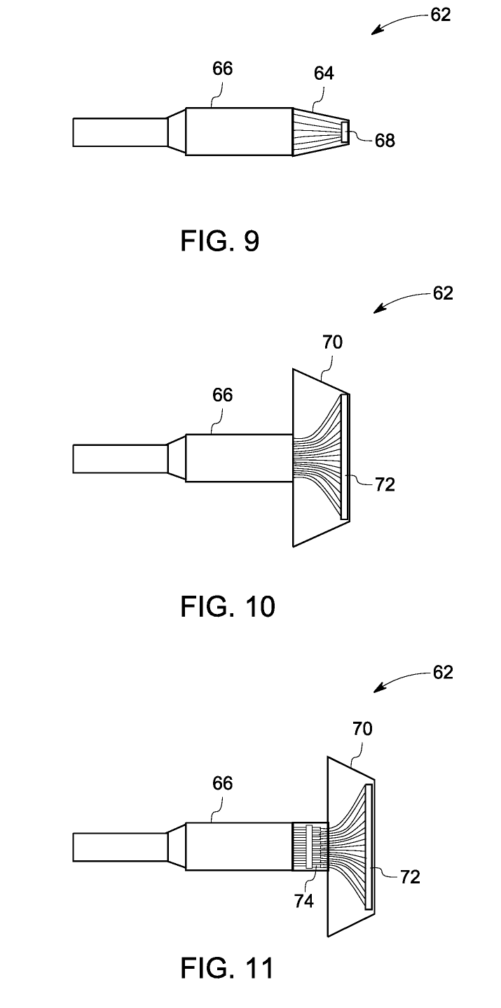

Referring to FIG. 9, an ultrasound probe 62 in accordance with an exemplary embodiment of the present invention is disclosed. In the illustrated embodiment, the probe 62 includes a head portion 64 and a handle portion 66 detachably coupled to the head portion 64. The handle portion 66 is detachably coupled to the head portion 64 via a mechanical joint. In the illustrated embodiment, a transducer 68 including a one or two-dimensional array of transducer elements is disposed in the head portion 64. It should be noted herein that the head portion 64 and the transducer 68 has a relatively smaller footprint. It should be noted herein that “footprint” refers to a patient contact surface of the head portion.

Referring to FIG. 10, an ultrasound probe 62 in accordance with another exemplary embodiment of the present invention is disclosed. In the illustrated embodiment, the probe 62 includes a head portion 70 and the handle portion 66 detachably coupled to the head portion 70. The handle portion 66 is detachably coupled to the head portion 70 via a mechanical joint. In the illustrated embodiment, a transducer 72 including a one or two-dimensional array of transducer elements is disposed in the head portion 70. It should be noted herein that the head portion 70 and the transducer 72 has a relatively larger footprint.

Referring to FIG. 11, an ultrasound probe 62 in accordance with another exemplary embodiment of the present invention is disclosed. The embodiment of FIG. 10 is similar to the embodiment discussed with reference to FIG. 9. Additionally, electronics module 74 may be disposed between the head portion 70 and the handle portion 66.

Referring to FIGS. 9, 10, 11, a probe is illustrated as having a detachable transducer head portion, whereby different transducer heads may be reversibly attached to the handle portion 66 of a common probe 62. The transducer head potions 64, 70 may have different dimensions, shapes and sizes depending on the particular imaging application required. For instance, smaller footprint transducer head portion 64 is used in applications requiring small acoustic windows, and larger footprint transducer head portion 70 is used in applications allowing larger acoustic windows. Additional electronics modules 74 may be disposed between the handle portion 66 and the transducer head portion 70. These electronics modules 74 may have functions including, but not limited to switching (multiplexing), amplifying, impedance matching, and beamforming. Electronic components (not shown) that enable the transducer head identification by the ultrasound system may also be included in the transducer head portions 64, 70.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An ultrasound probe, comprising:
a head portion,
a transducer comprising an array of transducer elements disposed in the head portion;
at least one or more stages of electronic circuit units coupled to the transducer and configured to excite the transducer;
a handle portion detachably coupled to the head portion; wherein the head portion and the handle portion are disposed enclosing the at least one or more stages of electronic circuit units;
wherein the ultrasound probe may be used for one dimensional applications, two dimensional applications, and volumetric applications.

2. The ultrasound probe of claim 1, wherein the electronic circuit unit comprises a modular electronic circuit unit.

3. The ultrasound probe of claim 2, wherein the modular electronic circuit unit comprises a first stage electronic circuit unit coupled to the transducer disposed in the head portion.

4. The ultrasound probe of claim 3, wherein the modular circuit unit comprises a second stage electronic circuit unit removably coupled to the first stage electronic circuit unit via a joint comprising an electrical joint, mechanical joint, or combinations thereof.

5. The ultrasound probe of claim 1, wherein the head portion is replaceable.

6. The ultrasound probe of claim 1, wherein the handle portion is detachably coupled to the head portion via a mechanical joint.

7. The ultrasound probe of claim 6, wherein the mechanical joint comprises a hook provided to the head portion and configured to be detachably coupled to one or more recesses provided in the handle portion.

8. The ultrasound probe of claim 6, further comprising a dielectric barrier disposed contacting the mechanical joint.

9. The ultrasound probe of claim 8, wherein the dielectric barrier comprises an O-ring seal.

10. A transducer stack assembly for an ultrasound probe, the transducer stack assembly, comprising:
at least one acoustic matching layer;
a dematching layer;
a piezoelectric transducer layer disposed between the at least one acoustic matching layer and the dematching layer;
an interposer layer; wherein the dematching layer is disposed on the interposer layer;
an integrated circuit comprising a plurality of conductive bumps, wherein the interposer layer is disposed between the dematching layer and the integrated circuit.

11. The assembly of claim 10, comprising two acoustic matching layers configured to propagate sound waves.

12. The assembly of claim 10, wherein the dematching layer is configured to isolate the interposer layer and the integrated circuit from acoustic energy.

13. The assembly of claim 10, wherein the conductive bumps comprises gold, copper, solder, silver epoxy, or combinations thereof.

14. A transducer stack assembly for an ultrasound probe, the transducer stack assembly, comprising:
at least one acoustic matching layer;
a dematching layer;
a piezoelectric transducer layer disposed between the at least one acoustic matching layer and the dematching layer;
a substrate provided with conductive bumps, wherein the dematching layer is disposed on the substrate provided with conductive bumps.

15. The assembly of claim 14, wherein the at least one acoustic matching layer is configured to propagate sound waves.

16. The assembly of claim 14, wherein the dematching layer is configured to isolate the substrate from acoustic energy.

17. A method, comprising:
detaching a head portion from a handle portion of an ultrasound probe;
replacing the detached head portion with another head portion;
coupling the replaced head portion detachably to the handle portion.

18. The method of claim 17, further comprising detaching a second stage electronic circuit unit from a first stage electronic circuit unit coupled to a transducer disposed in the detached head portion.

19. The method of claim 17, comprising coupling the replaced head portion detachably to the handle portion via a mechanical joint.

20. The method of claim 19, comprising engaging a hook provided to the head portion detachably to one or more recesses provided in the handle portion.

21. The method of claim 19, further comprising providing a dielectric barrier contacting the mechanical joint.

22. A method of manufacturing a transducer stack assembly for an ultrasound probe, the method comprising:
providing at least one acoustic matching layer;
providing a dematching layer;
disposing a piezoelectric transducer layer between the at least one acoustic matching layer and the dematching layer; and
disposing an interposer layer between the dematching layer and an integrated circuit; wherein an integrated circuit comprises a plurality of conductive bumps.

23. The method of claim 22, comprising providing two acoustic matching layers configured to propagate sound waves.

24. A method of manufacturing a transducer stack assembly for an ultrasound probe, the method comprising:
providing at least one acoustic matching layer;
disposing a piezoelectric transducer layer disposed between the at least one acoustic matching layer and a dematching layer; and
disposing the dematching layer on the substrate provided with conductive bumps.
